(19) United States
(12) Patent Application Publication
(10) Pub. No.: US 2007/0049820 A1
(43) Pub. Date: Mar. 1, 2007

(54) Remotely controllable needle guide

(76) Inventors: Dieter Stern, Forst (DE); Swen Kohn, Karlsruhe (DE); Lothar Gumb, Graben-Neudorf (DE)

Correspondence Address: KLAUS J. BACH, 4407 TWIN OAKS DRIVE, MURRYSVILLE, PA, 15668, US

(21) Appl. No.: 11/526,893

(22) Filed: Sep. 22, 2006

Related U.S. Application Data
(63) Continuation-in-part of application No. PCT/EP05/03132, filed on Mar. 24, 2005.

(30) Foreign Application Priority Data
Apr. 1, 2004 (DE) 10 2004 015 971.8

(51) Int. Cl. A61B 5/05

(52) U.S. Cl. 600/427; 604/131

(57) ABSTRACT

In a remotely controllable needle guide arrangement for the insertion of a long needle into a patient body under tight space conditions, at least one bent positive guide structure is provided for guiding the needle and the needle has elastic properties such that the needle, after passing the bent positive guide structure, the needle is fully straightened out again.

This is a continuation-in-part application of international application PCT/EP2005/003132 filed Mar. 24, 2005 and claiming the priority of German application 10 2004 015 971.8 filed Apr. 1, 2004.

### BACKGROUND OF THE INVENTION

The present invention resides in a remotely controllable needle guide structure for the insertion of long needles such as injection needles for the injection of medications, medical preparations or contrast media or for the introduction of instruments into the body of a patient under tight conditions 1.

The tight conditions mentioned are present for example in connection with interventional radiological examinations or operations for example in a computer tomography (CT) or magnetic resonance tomography (MRT). Generally, the patient is then disposed on a patient rest in the MRT- or CT channel while the treating physician supervises and controls the operation from the outside while observing it on a picture tube utilizing the MRT or CT data.

In operations of this type, for example medicines, medical preparations or contrast media are injected into a patient using long needles which are inserted into the patient by an imaging-supported procedure so that, under the tight conditions, they reach a predetermined target position. Straight needles facilitate a precise positioning of the needle tip in the body of the patient.

Alternatively, needle guides of the type referred to above are used for the introduction of optical, electrical, mechanical, or cryo-engineering connections (for example, cables, glass fibers, etc.) into the body of a patient.

DE 41 13 045 C1 discloses for example a needle guide for a straight needle which is arranged so as to be movable from the distal end through a plastic guide which is also straight.

This guide however has, under the tight conditions mentioned above, only a limited field of application. Particularly the channel walls of an MRT or a CT delimit the space around a patient so that relatively long needles can be inserted into the body of patient disposed in such a channel only in an inclined orientation.

It is therefore the object of the present invention to provide a needle guide which does not have the limitations mentioned above, but which facilitates an improved and more accurate positioning of a needle tip in the body of a patient.

### SUMMARY OF THE INVENTION

In a remotely controllable needle guide arrangement for the insertion of a long needle into a patient body under tight space conditions, at least one bent positive guide structure is provided for guiding the needle and the needle has elastic properties such that the needle, after passing the bent positive guide structure, the needle is fully straightened out again.

The positive needle guide structure includes at least one bent positive guide area for the needle. With such a bent positive guide area, a long needle can be moved up to the insertion point parallel to the patients body where it can then be re-oriented to be positioned at a predetermined angle with respect to the body. As needle therefore an elastic needle is used which, outside the bent positive guide area, resumes its original shape. As needle materials particularly super-elastic materials such as memory alloys are suitable which can be bent in the bent positive guide area also to relatively small radii and, as a result, reduce the necessary space requirements for the needle guide in an advantageous manner.

Preferably, the bent positive guide area comprises at least a guide hose which is fixed at both ends and in which the needle is axially movably guided. As guide hoses particularly resiliently bendable hoses with a form-stable cross-section and small friction resistance with respect to the needle are suit-able for example nylon hoses or Bowden control cable hoses with spiral wire walls. The two ends do not only serve the positioning but also the orientation of the needle leaving the hose at the respective end. Therefore, at least one of the two ends of each guide hose is adjustable in its position and orientation by way of control devices (piston drives, control motors, etc.) and, if applicable, a separate sensor structure (distance and angle sensors).

For the advancing and retracting of the needle in the bent positive guide area an advancing arrangement is provided, preferably a linear drive (control motor, control piston, etc.) with a gripper for grasping while advancing and retracting the needle. A most accurately positioned and oriented insertion of the needle into a patients body is achieved if the engagement location of the advancing arrangement is positioned at the distal end of the bent positive guide area as close as possible to the insertion point at the patient and is not affected by the bent positive guide area. With the grasper, a final orientation and positioning of the needle before the insertion point outside the patients body is facilitated.

The advancing arrangement works similar to the so-called stick-slip-principle. After the needle has been grasped, it is axially advanced or retracted and is released in the new position, in which it is held by the friction in the needle guide and in the patient's body. The released gripper is then moved relative to the needle and again grasps the needle at a different location.

Alternatively, so-called inch worm drives are proposed as advancing arrangements. They consist of two alternately engaging graspers for the needle with a linear actuator disposed therebetween for generating the advance movement. In a particular embodiment also the distal end gripper may be in the form of a guide gripper which may be uncoupled from the drive but is optionally provided with an additional directional correction capability.

In another alternative arrangement, basically also friction wheel drives may be used which however may result in an increased wear of the needle and, as a result, substantially limit their use for inserting a needle into the body of a patient.

All drives and advancing arrangements mentioned are non-manual and therefore basically remotely controllable.

As a medical apparatus, the needle guide must satisfy special requirements with regard to sterility. In particular, the needle guide comprises a sterile area for the needle which preferably is enclosed by the bent positive guide structure and by sterile protection hoses sealingly connected to the bent positive guide structure.

Further embodiments of the needle guide structure, such as the minimally invasive insertion for example of probes, instruments, electrodes, catheters, or actuators such as ultrasound transducers do not require basically different designs for the needle guide structure and are therefore also included in the invention at least as equivalents.

The invention will be explained in greater detail below with reference to the exemplary drawings.

### BRIEF DESCRIPTION OF THE DRAWINGS

FIG. 1 a simplified side view of an exemplary embodiment, and

FIG. 2 shows a needle with a single use component of the exemplary embodiment.

### DESCRIPTION OF A PARTICULAR EMBODIMENT

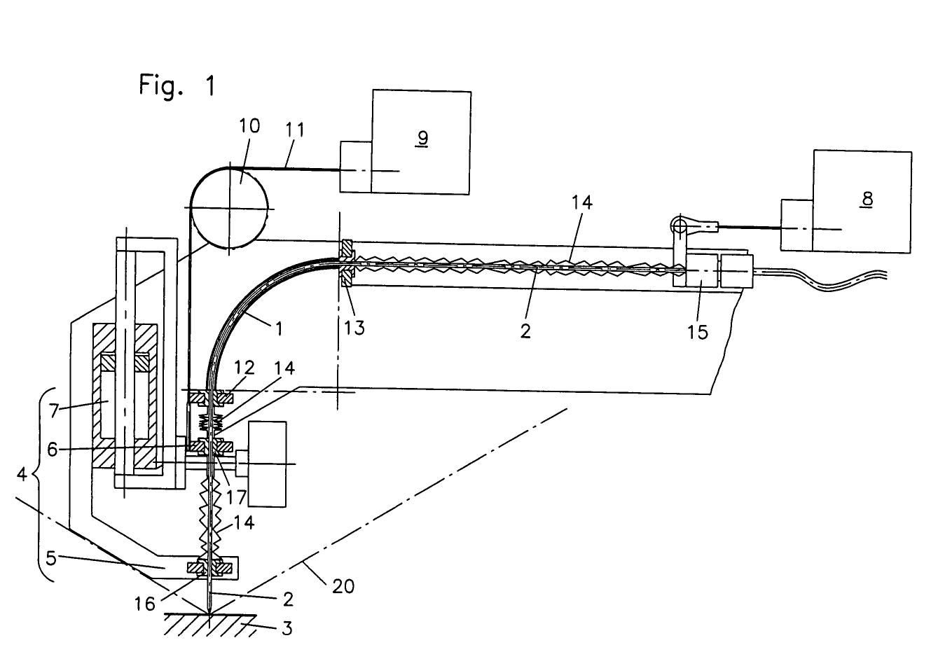

The exemplary embodiment shown in FIG. 1 comprises essentially a guide hose 1 for re-directing an elastic needle 2 toward the body of a patient 3. The advancing device 4 in the form of an inch worm drive engages the needle 2 by means of a pneumatic guide gripper 5 and a gripper 6, which is also pneumatically operated. The grippers are arranged at the distal end of the guide hose 1 as close as possible to the patient's body 3 and are alternately actuated. By their arrangement close to the patient's body, resiliency or friction effects in the needle guide structure affecting the positioning accuracy are reduced to a minimum. The advancing arrangement of FIG. 1 comprises furthermore a bi-directional pneumatic cylinder which serves as a linear actuator 7, wherein the stationary guide gripper 5 is connected to the cylinder and the movable gripper 6 is connected to the piston of the pneumatic cylinder drive. The pneumatic advancing arrangement does not require any magnetic or electrically conductive components so the drive arrangement can advantageously also be used in an MRT or, respectively, a CT.

In an advancement movement first the needle is engaged by the guide gripper 5, or it is held alone by the friction in the apparatus while the gripper 6 moves to its grasping position. In the grasping position, the needle is engaged by the gripper 6 and the guide gripper 5 is released and the needle is then moved by the gripper 6 using the linear drive 7 in a desired direction or to a desired position. During the advance movement, the guide gripper 5 and a clamping structure 12 serve advantageously as mechanical safety limits for the guide gripper 5. When a position has been reached, by releasing the gripper 6 and repositioning it on the needle, a further advance movement can be initiated by a repeat of the steps mentioned above.

The needle preferably consists of a super-elastic alloy, for example, an Ni—Ti alloy with the known mechanical and bio-compatible properties.

FIG. 1 furthermore shows a travel distance measuring system 8 for the advancement of the needle 2 and a position measuring system 9 for the linear actuator 7, which foam part of the sensing equipment. The position measuring system 9 comprises two-travel distance sensors for determining the position of the gripper 6. The transfer from the grippers to the position measuring system occurs via ropes 11 with a reversing roller 10.

FIG. 1 for example does not show a carrier element which serves as support structure and/or a housing for the components shown.

The guide hose 1 ends at each of its sides with a clamping piece 12 and 13, which is connected to the support element by a fixing structure, which are not shown. Particularly the distal end clamping piece 12 can be positioned and oriented by way of the fixing structure in a preferred embodiment via its own adjustment arrangements (drives, pneumatic cylinder, Bowden mechanism, etc.) together with the advancing arrangement 4 in at least one of the six degrees of freedom (3× lateral, 3× rotational). With respect to a possible MRT or, respectively, CT-suitably of this adjustment arrangement and the additional sensing equipment needed herefor, it is noted that the same considerations apply as mentioned for the advancing arrangement 4 (no electrically conductive or magnetic material generation of electric or magnetic fields).

The needle guide arrangement of FIG. 1 shows furthermore several sterile protection hoses 14 (thin-walled plastic hoses) which, together with the guide hose 1, form a sterile area for the needle 2. In the exemplary embodiment, a sterile protective hose bridges in each case the space between the two grippers 5 and 6, the gripper 6 and the clamping piece 12 and the clamping piece 13 and the Luer lock projection 15 at the proximal end of the needle 2.

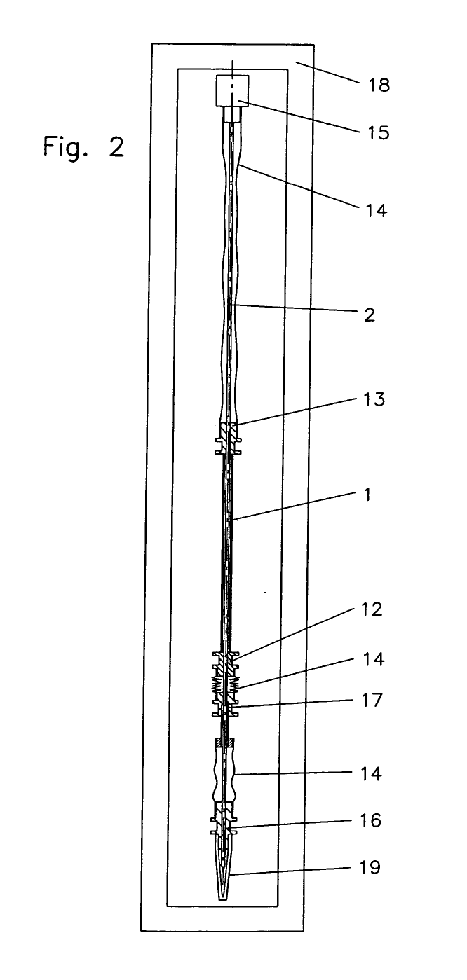

It would be possible to design all components which surround the sterile area together with the needle 2, the clamping pieces 12 and 13 as well as the two adapter members 16 and 17 for the two grippers 5 and 6 in the form of a contiguous assembly group as single use components. FIG. 2 shows for example this assembly group in a sterile packing 18 consisting of two plastic foils which can be welded together wherein the distal end needle point is covered by a protective cover 19.

For use, the sterile assembly (single use components with needle and sterile area) is removed from the sterile packing and prepared depending on its application. It is for example connected to a medication dosing device or it is prepared as guide tube for a laser light conductor, for cryo conduits, etc. Only subsequently, the assembly is installed in the apparatus (the second, non-sterile structure), wherein only with the jointure of the sterile and the non-sterile components the needle guide structure is formed.

From a medical point of view, it is more acceptable for a patient if, before being pierced by the needle via a remotely operated machine, he or she is contacted at the piercing location by a soft sterile contacting structure 20, which is lightly pressed against the patient (with a force in the area of 10 to 20 N) as indicated in FIG. 1. The patient contacting structure 20 in the embodiment presented comprises a cone-like compressible structure with a core of foam material which ensures that around the targeted piercing location the patient is contacted over an area and the tissue layers of the body of the patient which have to be pierced do not move relative to one another. FIG. 1 does not show a central cone-like opening for the needle in the center of the patient contacting structure.

What is claimed is:

1. A remotely controllable needle guide arrangement for the insertion of a long needle (2) into a patient's body (3) under tight space conditions, comprising at least one bent positive guide structure (1) for the needle (2) and the needle having elastic properties.

2. A remotely controllable needle guide arrangement according to claim 1, wherein the needle (2) has super-elastic properties.

3. A remotely controllable needle guide arrangement according to claim 1, wherein the bent positive guide structure comprises at least one guide hose (1) which is fixed at both ends and in which the needle (2) is axially movably guided.

4. A remotely controllable needle guide arrangement according to claim 3, including an adjustment arrangement with drive means and sensor devices wherein at least one of the two ends is adjustable with respect to its position and orientation.

5. A remotely controllable needle guide arrangement according to claim 1, comprising an advancing arrangement (4) for grasping and for advancing and retracting the needle (2).

6. A remotely controllable needle guide arrangement according to claim 5, wherein the advancing arrangement comprises a linear drive (7) and at least one gripper (6) arranged at the distal end of the bent positive guide structure (1).

7. A remotely controllable needle guide arrangement according to claim 1, further comprising a sterile area for the needle (2).

8. A remotely controllable needle guide arrangement according to claim 7, wherein the sterile area is surrounded by the bent positive guide structure and sterile protective hoses (14) disposed adjacent thereto and in sealed relationship therewith.

9. A remotely controllable needle guide arrangement according to claim 1 for examinations or operations in a computer tomography, CT, or in a magnetic resonance tomography, MRT, wherein the needle guide arrangement can be introduced into the CT or MRT because of its geometric dimensions and sensor and drive structures are operable therein, and all components of the needle guide structure consist of non-magnetic materials and furthermore the drives and sensor devices of the arrangement do not require, or generate, any magnetic or electrical fields.

10. A remotely controllable needle guide arrangement according to claim 8, wherein the needle and the components of the needle guide structure are divided into two design groups, wherein one design group comprises re-usable components and the other design group comprises single use components.

11. A remotely controllable needle guide arrangement according to claim 10, wherein the re-usable components are the adjustment arrangement and the sensor devices and the single use components comprise the needle, the bent positive guide structure and the sterile hoses.
